(19) United States
(12) Patent Application Publication
(10) Pub. No.: US 2020/0268058 A1
(43) Pub. Date: Aug. 27, 2020

(54) ELECTRONIC CIGARETTE WITH ADDITIONAL FUNCTIONS OF ALCOHOL TESTING AND WIRELESS COMMUNICATION

(71) Applicant: Goldtek Technology Co., Ltd., New Taipei City (TW)

(72) Inventors: PAI-CHOU CHIANG, New Taipei (TW); CHIEN-LUNG HUANG, New Taipei (TW); CHAO-SHEN CHEN, New Taipei (TW)

(21) Appl. No.: 16/373,115

(22) Filed: Apr. 2, 2019

(30) Foreign Application Priority Data
Feb. 27, 2019 (TW) 108106662

(51) Int. Cl. A24F 47/00; G01N 33/497; G01N 33/00

(52) U.S. Cl. CPC A24F 47/008; H04B 7/26; G01N 33/0063; G01N 33/4972

(57) ABSTRACT

An electronic cigarette able to test for alcohol on the breath and transmit such data by wire or wirelessly to another device for comparison purposes and for display includes a liquid module, an atomizer, an alcohol testing module, and a microprocessor. Tobacco tar from the liquid module is converted by the atomizer into smoke. The alcohol testing module detects user's alcohol concentration on the breath when the electronic cigarette is turned on. The microprocessor records all data and establishes wireless communication with a portable electronic device and/or a server. The operating data of the atomizer and the alcohol concentration data can be transmitted to the portable electronic device and/or the server for being analyzed and storage.

### FIELD

The present disclosure relates to an electronic cigarette, particularly relates to an electronic cigarette with additional functions of alcohol testing and wireless communication.

### BACKGROUND

Electronic cigarettes can replace the traditional cigarettes and help people to give up smoking. The electronic cigarettes have similar appearance and taste like traditional cigarettes. The electronic cigarette is formed by a shaft and an atomizer. The shaft includes battery and other kinds of circuits. However, the shaft is only a simple connection with the atomizer. Smoking and drinking are two activities that some say are harmful for humans, these two behaviors often happen at the same time. An alcohol testing function added to the shaft of the electronic cigarette would by useful. Wireless communication devices are also ubiquitous, thus an electronic cigarette adapted for wireless communication with wireless communication devices to exchange data may be useful and desired.

Thus, there is room for improvement within the art.

### BRIEF DESCRIPTION OF THE DRAWING

Many aspects of the disclosure can be better understood with reference to the following drawing. The components in the drawing are not necessarily drawn to scale, the emphasis instead being placed upon clearly illustrating the principles of the disclosure. Moreover, in the drawing, like reference numerals designate corresponding parts throughout the several views.

FIG. 1 is a functional block diagram of an embodiment of an electronic cigarette.

### DETAILED DESCRIPTION

In order to make the above-mentioned objects, features and advantages of the present implementation more obvious, a detailed description of specific embodiments of the present implementation will be described in detail with reference to the accompanying drawings. A number of details are set forth in the following description so as to fully understand the present implementation. However, the present implementation can be implemented in many other ways different from those described herein, and those skilled in the art can make similar improvements without violating the contents of the present implementation. Therefore, the present implementation is not to be considered as limiting the scope of the embodiments described herein.

Several definitions that apply throughout this disclosure will now be presented.

Unless otherwise defined, all technical and scientific terms used herein have the same meaning as commonly understood by one skilled in the art. The terms used in a specification of the present implementation herein are only for describing specific embodiments, and are not intended to limit the present implementation. The terms “and/or” used herein includes any and all combinations of one or more of associated listed items.

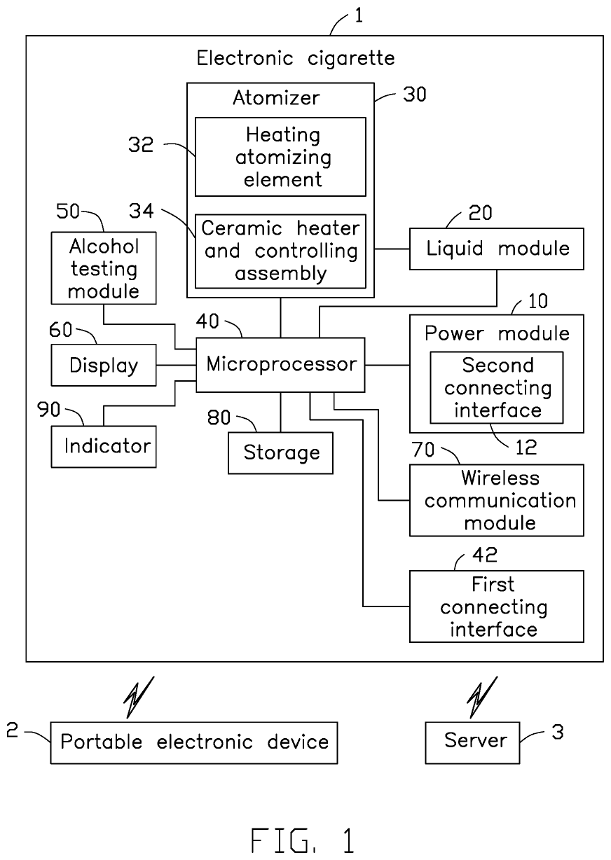

FIG. 1 shows one embodiment of an electronic cigarette 1. The electronic cigarette 1 includes a power module 10, a liquid module 20, an atomizer 30, a microprocessor 40, an alcohol testing module 50, a display 60, a wireless communication module 70, a storage 80, an indicator 90, and a first connecting interface 42. The electronic cigarette 1 further includes a housing (not shown) or a shaft (not shown) configured for receiving all the aforesaid electronic elements and modules. The housing or the shaft can be shaped as required. The housing or the shaft may further include buttons (not shown), such as power button (not shown) and buttons for adjusting operations (not shown) of the atomizer 30. In at least one embodiment, the electronic cigarette 1 is adapted for establishing wireless communication with a portable electronic device 2 to exchanging data to and from the electronic cigarette 1. The portable electronic device 2 can be but is not limited to a mobile phone, a table computer, a personal digital assistant (PDA), and a notebook computer.

The power module 10 provides power for the electronic cigarette 1. In at least one embodiment, the power module 10 is a rechargeable battery. The power module 10 includes a second connecting interface 12 for electrically connecting to an external power source (not shown) through a transmission cable (not shown) for recharging. In addition, the power module 10 may electrically connects to the portable electronic device 2 through the transmission cable to share electrical current to recharge the portable electronic device 2. In at least one embodiment, the second connecting interface 12 can be but is not limited to a USB interface.

The liquid module 20 is configured to contain tobacco tar/atomizer liquid. The atomizer 30 converts the tobacco tar/atomizer liquid into smoke. The atomizer 30 includes a heating atomizing element 32 and a ceramic heater and controlling assembly 34. The heating atomizing element 32 and the ceramic heater and controlling assembly 34 are two kinds of atomizing elements with similar functions, therefore, the heating atomizing element 32 and the ceramic heater and controlling assembly 34 are interchangeable. The electronic cigarette 1 can selectively set one of the heating atomizing element 32 or the ceramic heater and controlling assembly 34 as the atomizer.

The microprocessor 40 is electrically connected to each of the power module 10, the liquid module 20, the atomizer 30, the alcohol testing module 50, the display 60, the wireless communication module 70, the storage 80, the indicator 90, and the first connecting interface 42. The microprocessor 40 controls the above-mentioned elements and modules and processes data of the above-mentioned elements and modules. Specifically, the microprocessor 40 detects the atomizing element (one of the heating atomizing element 32 and the ceramic heater and controlling assembly 34) set to the atomizer 30. In at least one embodiment, the microprocessor 40 generates operating data, such as data of an operating power of the atomizing element, according to the atomizing element set to the atomizer 30. The microprocessor 40 is further configured to record the operating data of the atomizer 30, such as data of an operating duration, an operating power, and a usage of the tobacco tar/atomizer liquid, thereby recording information of smoking habit of the user smoking through the electronic cigarette 1.

The alcohol testing module 50 detects an alcohol concentration of the breath when the user is smoking. In at least one embodiment, the alcohol testing module 50 includes an alcohol sensor configured to detect the alcohol concentration of the breath when the user is smoking. The microprocessor 40 collects and records the alcohol concentration data detected by the alcohol testing module 50, and stores the alcohol concentration data to the storage 80.

The display 60 displays the operating data of the atomizer 30 and the alcohol concentration data recorded by the microprocessor 40. The operating data of the atomizer 30 includes but is not limited to the operating duration, the operating power, and the usage of the tobacco tar/atomizer liquid. Additionally, the display 60 can display current time, a power usage of the power module 10, and other data.

The wireless communication module 70 is adapted for establishing wireless communication connection with the portable electronic device 2. The microprocessor 40 transmits the operating data of the atomizer 30 and the alcohol concentration data recorded by the microprocessor 40 to the portable electronic device 2 through the wireless communication module 70. In at least one embodiment, the wireless communication module 70 is adapted for establishing wireless communication such as, but is not limited to, Sub 1 GHz, BLUETOOTH Low Energy (BLE), WI-FI, Narrow Band Internet of Things (NB-IoT), Long Range (LoRa), and LTE-Machine to Machine (LTE-M). In another embodiment, the wireless communication module 70 can establish wireless communication connection with a server 3. The microprocessor 40 transmits the operating data of the atomizer 30 and the alcohol concentration data recorded by the microprocessor 40 to the server 3 for storage.

The storage 80 stores the operating data of the atomizer 30 and the alcohol concentration data recorded by the microprocessor 40.

The indicator 90 indicates operating status of the electronic cigarette 1. In at least one embodiment, the indicator 90 can be but, is not limited to, an indicator light. When the electronic cigarette 1 is activated, the indicator light is on.

The first connecting interface 42 can be electrically connected to the portable electronic device 2 through a transmission cable (not shown). The microprocessor 40 transmits the operating data of the atomizer 30 and the alcohol concentration data recorded by the microprocessor 40 to the portable electronic device 2 through the first connecting interface 42 and the transmission cable. In at least one embodiment, the electronic cigarette 1 can be in wireless communication with the portable electronic device 2 through the wireless communication module 70 or through the transmission cable electrically connected with the portable electronic device 2 via the first connecting interface 42.

The portable electronic device 2 receives the operating data of the atomizer 30 and the alcohol concentration data from the electronic cigarette 1, and can display said data. In addition, the portable electronic device 2 analyzes the alcohol concentration data and compares the alcohol concentration data against an alcohol concentration standard value. When the alcohol concentration data is greater than the alcohol concentration standard value, the portable electronic device 2 generates a warning signal. In at least one embodiment, the form of the warning signal can be but is not limited to vibration, sound, text message, etc. In another embodiment, the warning signal can be outputted by the electronic cigarette 1. The portable electronic device 2 transmits warning information to the electronic cigarette 1, the electronic cigarette 1 receives the warning information through the wireless communication module 70 or through the transmission cable via the first connecting interface 42 and the microprocessor 40 controls the indicator 90 to output the warning signal. The form of the warning signal can be continuous flashing of the indicator light or a text message on the display 60.

In another embodiment, the electronic cigarette 1 may include other sensors, such as sensors for detecting glycemic index, blood oxygen level, pulmonary condition, heartbeat, or for analyzing saliva.

In at least one embodiment, the portable electronic device 2 may include a processor (not shown) and/or an analyzing module (not shown). The processor and/or the analyzing module are installable in the portable electronic device 2 for analyzing the operating data of the atomizer and the alcohol concentration data of the electronic cigarette 1.

In the present disclosure, the electronic cigarette 1 provides an electronic cigarette for the user and the alcohol testing module 50 of the electronic cigarette 1 detects alcohol concentration data of the breath of the user and analyzes the alcoholicity of the breath of the user. In addition, the electronic cigarette 1 transmits the operating data of the atomizer 30 and the alcohol concentration data to the portable electronic device 2 and the server 3 for storage and easy viewing by the user. Therefore, the electronic cigarette 1 is multi-functional and can improve a user experience especially after heavy drinking.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. An electronic cigarette, comprising:
a liquid module configured to contain tobacco tar;
an atomizer converting the tobacco tar in the liquid module into smoke;
an alcohol testing module detecting an alcohol concentration of breath when the electronic cigarette is turned on; and
a microprocessor recording operating data of the atomizer and alcohol concentration data detected by the alcohol testing module;
wherein the electronic cigarette establishes wireless communication with a portable electronic device and/or a server, the microprocessor transmits the operating data of the atomizer and the alcohol concentration data to the portable electronic device and/or the server for being analyzed and stored.

2. The electronic cigarette of claim 1, further comprising a wireless communication module, wherein the electronic cigarette establishes wireless communication with the portable electronic device and/or the server through the wireless communication module, and transmits the operating data of the atomizer and the alcohol concentration data to the portable electronic device and/or the server through the wireless communication module.

3. The electronic cigarette of claim 2, wherein the wireless communication module is adapted for establishing wireless communications in Sub 1 GHz, Bluetooth Low Energy (BLE), WiFi, Narrow Band Internet of Things (NB-IoT), Long Range (LoRa), LTE-Machine to Machine (LTE-M).

4. The electronic cigarette of claim 3, further comprising an indicator configured for indicating an operating status of the electronic cigarette.

5. The electronic cigarette of claim 4, wherein a processor and/or an analyzing module installable in the portable electronic device, when the portable electronic device installs the processor and/or the analyzing module, the processor and/or the analyzing module enables the portable electronic device to analyze the alcohol concentration data and compares to an alcohol concentration standard value, if the alcohol concentration data is greater than the alcohol concentration standard value, the electronic cigarette receives warning information from the portable electronic device through the wireless communication module, the microprocessor controls the indicator to output a warning signal.

6. The electronic cigarette of claim 5, further comprising a first connecting interface, wherein the first connecting interface is configured to electrically connect the portable electronic device to the electronic cigarette, the electronic cigarette transmits the operating data of the atomizer and the alcohol concentration data to the portable electronic device through the first connecting interface, and receives the warning information from the portable electronic device through the first connecting interface.

7. The electronic cigarette of claim 1, further comprising a power module adapted for providing electrical power to the electronic cigarette, wherein power module is a rechargeable battery, the power module comprises a second connecting interface adapted for electrically connecting to an external power source through a transmission cable to be recharged, the power module is further adapted for electrically connecting to the portable electronic device through the transmission cable to output electric power to the portable electronic device.

8. The electronic cigarette of claim 1, wherein the atomizer comprises a heating atomizing element, and a ceramic heater and controlling assembly, the electronic cigarette selects and arranges the atomizer from one of the heating atomizing element and the ceramic heater and controlling assembly; the microprocessor detects a selected and arranged atomizer, and generates operating data of the selected and arranged atomizer, the operating data of the selected and arranged atomizer comprises operating duration, an operating power, and a usage of the tobacco tar of the electronic cigarette.

9. The electronic cigarette of claim 1, further comprising a display adapted for displaying the operating data of the atomizer, the alcohol concentration data, and a usage of electric power of the electronic cigarette.

10. The electronic cigarette of claim 1, further comprising a storage adapted for storing the operating data of the atomizer and the alcohol concentration data.
